(19) United States
(12) Patent Application Publication
(10) Pub. No.: US 2025/0111514 A1
(43) Pub. Date: Apr. 3, 2025

(54) MEDICAL IMAGE PROCESSING APPARATUS, MEDICAL IMAGE PROCESSING METHOD, AND STORAGE MEDIUM STORING MEDICAL IMAGE PROCESSING PROGRAM

(71) Applicant: CANON MEDICAL SYSTEMS CORPORATION, Otawara-shi (JP)

(72) Inventors: Ryo SHIROISHI, Kawasaki (JP); Takuya SAKAGUCHI, Utsunomiya (JP)

(73) Assignee: CANON MEDICAL SYSTEMS CORPORATION, Otawara-shi (JP)

(21) Appl. No.: 18/904,791

(22) Filed: Oct. 2, 2024

(30) Foreign Application Priority Data
Oct. 3, 2023 (JP) 2023-171823

(51) Int. Cl. G06T 7/00; A61B 6/00; A61B 6/12; G06T 7/60

(52) U.S. Cl. CPC G06T 7/0014; A61B 6/12; A61B 6/487; G06T 7/60; G06T 2207/10121; G06T 2207/30101

(57) ABSTRACT

A medical image processing apparatus according includes processing circuitry. The processing circuitry is configured to calculate, from a plurality of medical X-ray fluoroscopic images, a size of a thrombus removal device that has been placed in a blood vessel of a subject and appears in the medical X-ray fluoroscopic image. The processing circuitry is configured to calculate change information indicating a change in the size from the plurality of medical X-ray fluoroscopic images. The processing circuitry is configured to calculate first time information indicating a time period during which the thrombus removal device is left in place in the subject, based on the change information. The processing circuitry is configured to perform control to output the first time information.

### CROSS-REFERENCE TO RELATED APPLICATIONS

This application is based upon and claims the benefit of priority from Japanese Patent Application No. 2023-171823, filed Oct. 3, 2023, the entire contents of which are incorporated herein by reference.

### FIELD

Embodiments according to the subject application relate generally to a medical image processing apparatus, a medical image processing method, and a storage medium storing a medical image processing program.

### BACKGROUND

As treatment for infarctions in which blood flow is obstructed due to occlusion of a blood vessel by a thrombus, there is a thrombectomy treatment for retrieving the thrombus by insertion of a catheter, a stent, and the like into the blood vessel. Infarctions may occur irrespective of a site and an organ of a human body. As an example of the thrombectomy treatment among those, there is a treatment for retrieving the thrombus by using a stent thrombus removal device, such as a stent retriever, for ischemic stroke in which infarction occurs in a cerebral blood vessel.

The stent retriever, which is a stent directed to removing a thrombus, is deployed from a catheter to a site where the thrombus is present, and is retrieved into a guide catheter so as to entangle the thrombus. At this time, in order to cause the stent retriever to sufficiently engage the thrombus, the stent retriever is left in the blood vessel for a predetermined recommended waiting time (e.g., five minutes) after the stent retriever is deployed in the blood vessel, and is then retrieved.

The recommended waiting time in using the thrombus removal device is, for example, a time until the thrombus is sufficiently entangled such that waiting any longer would not result in further engagement of the thrombus. A suitable waiting time depends on a case because an amount, a shape, and the like of a thrombus are different between cases. To retrieve the thrombus after the waiting time suitable for the case, it is desirable that a suitable waiting time be estimated from case-specific observation data, and the suitable waiting time be presented to an operator as a recommended waiting time.

If a state of the thrombus can be grasped with each moment in a process in which the thrombus is gradually removed during the thrombectomy treatment, the recommended waiting time can be easily determined from time-series data on the thrombus by using existing techniques such as linear regression and nonlinear regression.

However, the thrombus cannot be visualized in an X-ray fluoroscopic image, and the amount and the shape thereof cannot be observed. When the X-ray fluoroscopy is continuously performed over a recommended waiting time, only intermittent time-series data is obtained because of a large exposure dose to the subject. Therefore, it is not easy to estimate a suitable waiting time from the case-specific observation data and to present the recommended waiting time to the operator, by using the existing techniques.

### BRIEF DESCRIPTION OF THE DRAWINGS

FIG. 1 is a diagram illustrating an exemplary configuration of a medical image processing system according to an exemplary embodiment;

FIG. 2 is a diagram illustrating an exemplary configuration of a medical image processing apparatus according to an exemplary embodiment;

FIG. 3 is a schematic diagram illustrating thrombus removal by using a thrombus removal device according to an exemplary embodiment;

FIG. 4 is a schematic diagram illustrating an example to explain a size of the thrombus removal device according to an exemplary embodiment;

FIG. 5 is a graph illustrating an example of a change in size of the thrombus removal device according to an exemplary embodiment;

FIG. 6 is a flowchart illustrating an exemplary processing procedure to be performed by the medical image processing apparatus according to an exemplary embodiment;

FIG. 7 is a graph illustrating an example of a change in size of a thrombus removal device according to a first modification;

FIG. 8 is a diagram illustrating an exemplary configuration of a medical image processing apparatus according to a second modification;

FIG. 9 is a graph illustrating an example to explain an imaging timing according to the second modification; and

FIG. 10 is a diagram illustrating an exemplary configuration of a medical image processing apparatus according to a third modification.

### DETAILED DESCRIPTION

A medical image processing apparatus according includes processing circuitry. The processing circuitry is configured to calculate, from a plurality of medical X-ray fluoroscopic images, a size of a thrombus removal device that has been placed in a blood vessel of a subject and appears in the medical X-ray fluoroscopic image. The processing circuitry is configured to calculate change information indicating a change in the size from the plurality of medical X-ray fluoroscopic images. The processing circuitry is configured to calculate first time information indicating a time period during which the thrombus removal device is left in place in the subject, based on the change information. The processing circuitry is configured to perform control to output the first time information.

Various Embodiments will be described hereinafter with reference to the accompanying drawings.

### Exemplary Embodiments

An exemplary embodiment is initially described. In the exemplary embodiment, an example in a case where a technique discussed in the subject application is applied to a medical image processing apparatus is described. In the following, a medical image processing system including the medical image processing apparatus is described as an example.

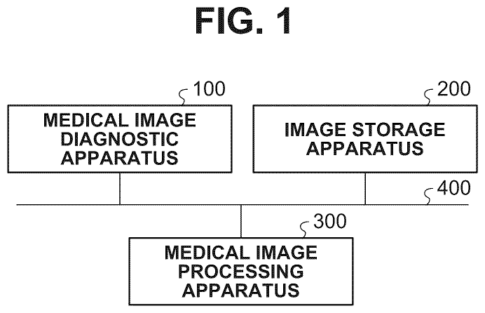

FIG. 1 is a diagram illustrating an exemplary configuration of the medical image processing system according to the exemplary embodiment. As illustrated in FIG. 1, the medical image processing system according to the exemplary embodiment includes a medical image diagnostic apparatus 100, an image storage apparatus 200, and a medical image processing apparatus 300.

For example, in the medical image processing system, the medical image processing apparatus 300 according to the exemplary embodiment is connected to the medical image diagnostic apparatus 100 and the image storage apparatus 200 through a network 400 as illustrated in FIG. 1.

The medical image diagnostic apparatus 100 is a modality used for a procedure using a medical device, and is, for example, an X-ray diagnostic apparatus, an X-ray computed tomography (CT) apparatus, an ultrasonic diagnostic apparatus, or a magnetic resonance imaging (MRI) apparatus. For example, the medical image diagnostic apparatus 100 collects projection data by imaging a subject, and collects two-dimensional or three-dimensional medical image data based on the collected projection data.

The medical image diagnostic apparatus 100 can collect two-dimensional or three-dimensional contrast image data by imaging a subject injected with a contrast agent. For example, the X-ray CT apparatus serving as the medical image diagnostic apparatus 100 collects CT angiography (CTA) image data from the subject injected with the contrast agent, as the three-dimensional contrast image data. For example, the X-ray diagnostic apparatus serving as the medical image diagnostic apparatus 100 collects X-ray image data from the subject injected with the contrast agent, as the two-dimensional contrast image data.

The medical image diagnostic apparatus 100 transmits the collected medical image data to the image storage apparatus 200 and/or the medical image processing apparatus 300. In transmitting the medical image data to the image storage apparatus 200 and/or the medical image processing apparatus 300, the medical image diagnostic apparatus 100 transmits, as accompanying information, for example, a patient identification (ID) for identification of a patient, an examination ID for identification of an examination, an apparatus ID for identification of the medical image diagnostic apparatus 100, a series ID for identification of single imaging performed by the medical image diagnostic apparatus 100, and other types of information.

The image storage apparatus 200 stores the medical image data collected by the medical image diagnostic apparatus 100 through the network 400. For example, the image storage apparatus 200 is realized by a computer apparatus such as a server apparatus. In the present exemplary embodiment, the image storage apparatus 200 acquires the two-dimensional or three-dimensional medical image data from the medical image diagnostic apparatus 100 through the network 400, and stores the acquired medical image data in storage circuitry provided inside the apparatus or outside the apparatus.

The medical image processing apparatus 300 acquires the medical image data from the medical image diagnostic apparatus 100 and the image storage apparatus 200 through the network 400, and processes the acquired medical image data. For example, the medical image processing apparatus 300 is realize by a computer apparatus, such as a workstation.

In the present exemplary embodiment, the medical image processing apparatus 300 acquires the medical image data from the medical image diagnostic apparatus 100 or the image storage apparatus 200 through the network 400, and performs various types of image processing on the acquired medical image data. Further, the medical image processing apparatus 300 displays a medical image having subjected to image processing, an analysis result obtained through the image processing, and the like on a display or the like.

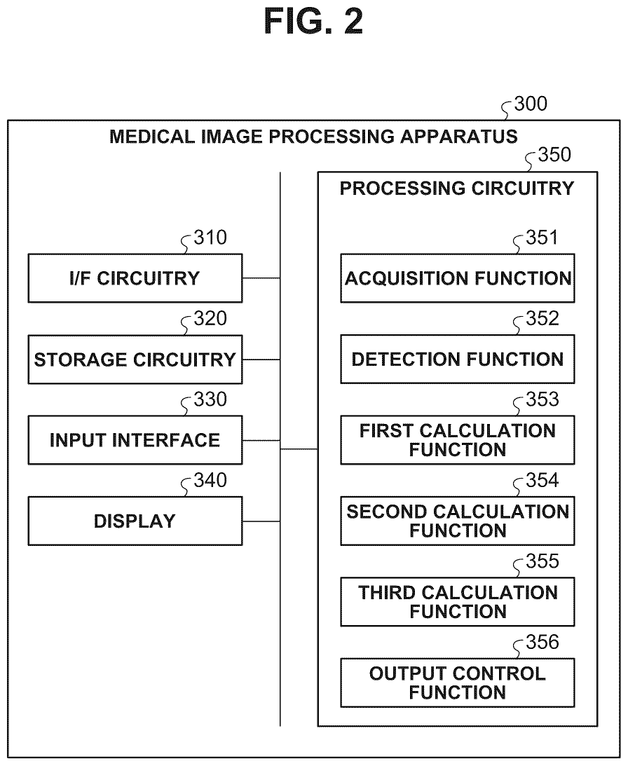

FIG. 2 is a diagram illustrating an exemplary configuration of the medical image processing apparatus 300 according to the present exemplary embodiment. For example, as illustrated in FIG. 2, the medical image processing apparatus 300 includes interface (I/F) circuitry 310, storage circuitry 320, an input interface 330, a display 340, and processing circuitry 350.

The I/F circuitry 310 is connected to the processing circuitry 350, and controls transmission of various types of data and communication performed with the medical image diagnostic apparatus 100 or the image storage apparatus 200 connected through the network 400. For example, the I/F circuitry 310 is realized by a network card, a network adaptor, and a network interface controller (NIC).

In the present exemplary embodiment, the I/F circuitry 310 receives the medical image data from the medical image diagnostic apparatus 100 or the image storage apparatus 200, and outputs the received medical image data to the processing circuitry 350. The I/F circuitry 310 can receive real-time medical image data collected by the medical image diagnostic apparatus 100, and output the real-time medical image data to the processing circuitry 350.

The storage circuitry 320 is connected to the processing circuitry 350, and stores various types of data. For example, the storage circuitry 320 is realized by a semiconductor memory element, such as a random access memory (RAM) or a flash memory, a hard disk, an optical disc, or the like. In the present exemplary embodiment, the storage circuitry 320 stores the medical image data received from the medical image diagnostic apparatus 100 or the image storage apparatus 200. For example, the storage circuitry 320 stores CT image data in multiple time phases obtained by the X-ray CT apparatus, X-ray image data collected by the X-ray diagnostic apparatus, and ultrasonic image data collected by the ultrasonic diagnostic apparatus. As an example, the storage circuitry 320 stores CTA image data, X-ray image data, and the like collected by imaging the subject injected with the contrast agent.

The storage circuitry 320 stores various types of information to be used for processing of the processing circuitry 350, a result of processing performed by the processing circuitry 350, or the like. For example, the storage circuitry 320 stores correspondence information to be referred by the processing circuitry 350. Details of the correspondence information are described below.

The input interface 330 is realized by, for example, a track ball for performing setting of a predetermined region (e.g., site to be treated), a switch button, a mouse, a keyboard, a touch pad with which a user performs an input operation by touching on an operation surface, a touch screen with a display screen and a touch pad integrated, non-contact input circuitry including an optical sensor, sound input circuitry, and a foot switch for the application of X-rays.

The input interface 330 is connected to the processing circuitry 350, converts input operation received from an operator into an electric signal, and outputs the electric signal to the processing circuitry 350. Herein, the input interface 330 is not limited to one including physical operation parts, such as a mouse and a keyboard. For example, electric signal processing circuitry that receives an electric signal corresponding to input operation from an external input device provided separately from the apparatus, and outputs the electric signal to control circuitry is also included in examples of the input interface.

The display 340 is connected to the processing circuitry 350, and displays various types of information and various images output from the processing circuitry 350. For example, the display 340 is realized by a liquid crystal monitor, a cathode ray tube (CRT) monitor, or a touch panel. For example, the display 340 displays a graphical user interface (GUI) for receiving an instruction from the operator, various images, and results of various types of processing performed by the processing circuitry 350.

The processing circuitry 350 controls the components included in the medical image processing apparatus 300 in response to the input operation received from the operator through the input interface 330. For example, the processing circuitry 350 is realized by a processor. In the present exemplary embodiment, the processing circuitry 350 causes the storage circuitry 320 to store the medical image data output from the I/F circuitry 310. The processing circuitry 350 reads out the medical image data from the storage circuitry 320, and causes the display 340 to display a medical image generated from the read medical image data. The processing circuitry 350 performs various types of calculation processing on the medical image data, and causes the display 340 to display a calculation result.

With such a configuration, the medical image processing apparatus 300 according to the present exemplary embodiment can provide a recommended waiting time of a thrombus removal device in a blood vessel of a subject, in accordance with a case. More specifically, the medical image processing apparatus 300 calculates, from each of a plurality of medical, a size of the thrombus removal device that has been placed in the blood vessel of the subject and appears in the medical X-ray fluoroscopic image, calculates change information indicating a change in the size from the plurality of medical, calculates and outputs first time information indicating a time when the thrombus removal device in the subject is to be retrieved, based on the change information, and informs an operator about a recommended waiting time period during which the thrombus removal device is left in place in the blood vessel of the subject, in accordance with the case.

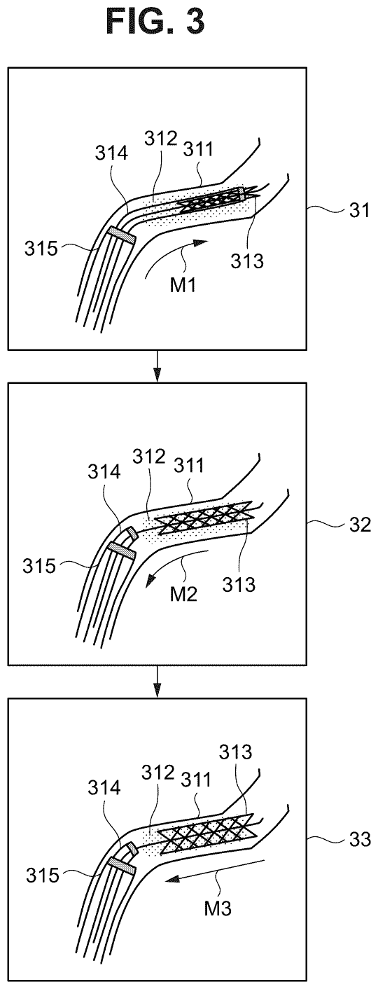

Here, a description will be provided of a method with which the thrombus removal device removes (retrieves) a thrombus from an inside of the blood vessel of the subject. For specific description, a stent retriever is described as an example of the thrombus removal device, and a cerebral blood vessel is described as an example of a blood vessel of the subject. FIG. 3 is a schematic diagram illustrating thrombus removal using the thrombus removal device according to the exemplary embodiment.

FIG. 3 illustrates a first medical X-ray fluoroscopic image 31, a second medical X-ray fluoroscopic image 32, and a third medical X-ray fluoroscopic image 33. In the first medical X-ray fluoroscopic image 31, the second medical X-ray fluoroscopic image 32, and the third medical X-ray fluoroscopic image 33, a blood vessel 311, a thrombus 312 present in the blood vessel 311, a stent retriever 313 which is a medical device for removing the thrombus 312, a microcatheter 314 which is a medical device for placing the stent retriever 313 in an inside of the blood vessel 311, and a guide catheter 315 which is a medical device for inserting the microcatheter 314 into the blood vessel 311 are illustrated.

Initially, to place the stent retriever 313 in the inside of the blood vessel 311, the operator inserts the guide catheter 315 from an artery of a lower limb of the subject. When the guide catheter 315 reaches a vicinity of the thrombus 312, the operator inserts the microcatheter 314 into the blood vessel 311 toward the thrombus 312 along a trajectory M1, and places the microcatheter 314 (see first medical X-ray fluoroscopic image 31 illustrated in FIG. 3).

After the operator places the microcatheter 314, the operator moves the microcatheter 314 to a position of the guide catheter 315 along a trajectory M2 (see second medical X-ray fluoroscopic image 32 illustrated in FIG. 3). This enables the stent retriever 313 to capture and secure the thrombus 312.

Subsequently, when the stent retriever 313 is deployed inside the blood vessel 311 and engages the thrombus 312, the operator moves the stent retriever 313 to the position of the guide catheter 315 along a trajectory M3 in order to retrieve the stent retriever 313 entangled with the thrombus 312 (see third medical X-ray fluoroscopic image 33 illustrated in FIG. 3). Thereafter, the operator retrieves the guide catheter 315 from the artery of the lower limb of the subject. This enables retrieval of the thrombus 312 present in the blood vessel 311 of the subject.

Thus, the operator can retrieve the thrombus 312 present in the blood vessel 311 of the subject by using the thrombus removal device; however, a time until the thrombus removal device is deployed (hereinafter, also referred to as recommended waiting time) depends on a case, and a method of determining a suitable recommended waiting time has not been established. Therefore, the medical image processing apparatus 300 according to the present exemplary embodiment provides a recommended waiting time during which the thrombus removal device is left in place in the blood vessel of the subject in accordance with a case.

Referring back to FIG. 2, the processing circuitry 350 of the medical image processing apparatus 300 according to the present exemplary embodiment performs an acquisition function 351, a detection function 352, a first calculation function 353, a second calculation function 354, a third calculation function 355, and an output control function 356.

The acquisition function 351 acquires a plurality of medical X-ray fluoroscopic images. The acquisition function 351 is an example of an acquisition unit. More specifically, the acquisition function 351 acquires the plurality of medical X-ray fluoroscopic images collected after the thrombus removal device is placed, from the medical image diagnostic apparatus 100 or the image storage apparatus 200 through the I/F circuitry 310.

The detection function 352 detects, from each of the plurality of medical X-ray fluoroscopic images, the thrombus removal device that has been placed inside the blood vessel of the subject and appears in the medical X-ray fluoroscopic image. The detection function 352 is an example of a detection unit. More specifically, the detection function 352 detects, from each of the plurality of medical X-ray fluoroscopic images acquired by the acquisition function 351, the thrombus removal device that has been placed inside the blood vessel of the subject and appears in the medical X-ray fluoroscopic images.

For example, in a case where the thrombus removal device is a stent retriever, the stent retriever is configured with a radiopaque material having a high X-ray absorption coefficient. For example, the detection function 352 performs threshold processing for masking a region where a pixel value in a medical X-ray fluoroscopic image is a predetermined value or less, on the plurality of medical X-ray fluoroscopic images acquired by the acquisition function 351, generates a plurality of device mask images, and detects the stent retriever.

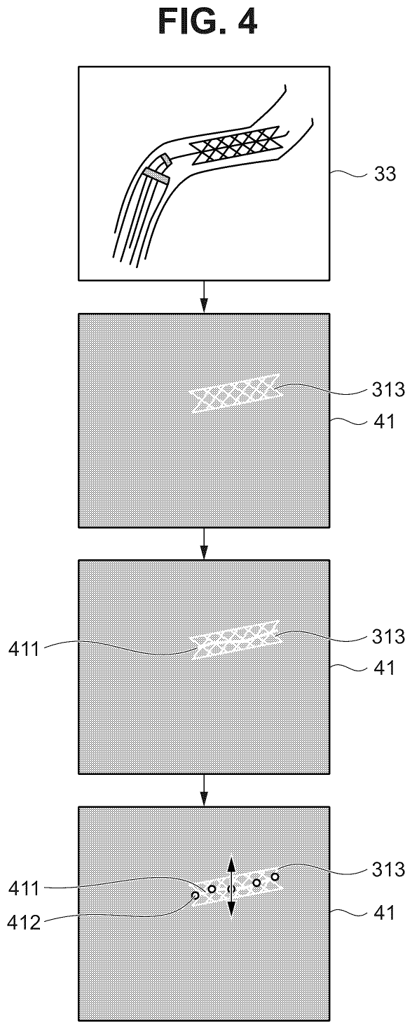

The first calculation function 353 calculates, from each of the plurality of medical X-ray fluoroscopic images, a size of the thrombus removal device that is placed inside the blood vessel of the subject and appears in the medical X-ray fluoroscopic image. The first calculation function 353 is an example of a first calculation unit. More specifically, the first calculation function 353 calculates a size of the thrombus removal device detected by the detection function 352. Here, the size of the thrombus removal device calculated by the first calculation function 353 is described with reference to FIG. 4. FIG. 4 is a schematic diagram illustrating an example for explanation of the size of the thrombus removal device according to the exemplary embodiment.

FIG. 4 illustrates the third medical X-ray fluoroscopic image 33 acquired by the acquisition function 351, and a device mask image 41 generated by the detection function 352. As described above, for example, the detection function 352 performs the threshold processing for masking a region where a pixel value in a medical X-ray fluoroscopic image is a predetermined value or less, on the plurality of medical X-ray fluoroscopic images acquired by the acquisition function 351, generates the device mask image 41, and detects the stent retriever 313.

The first calculation function 353 calculates a volume of a column with the stent retriever 313 present in the detected device mask image serving as a projection cross- section. For example, the first calculation function 353 fits a Bezier curve 411 to the device mask image 41 illustrated in FIG. 4.

The first calculation function 353 sets points with equal intervals on the Bezier curve 411, calculates a length 412 of the device in both normal directions of each of the points, averages the lengths 412, and determines the average to be a radius of the column at each of the points. Further, the first calculation function 353 calculates the volume of the column (lumen volume of stent retriever 313) with the radius at each of the points with the Bezier curve 411 as a center axis.

The above-described method is not restrictive. The stent retriever 313 can be detected with other methods, and the lumen volume of the stent retriever 313 can be calculated with other methods. For example, the device mask image may be generated by a stent retriever detection model that has trained a combination of a medical X-ray fluoroscopic image in which the stent retriever is imaged, and positional information about the stent retriever in the image.

For example, the lumen volume of the stent retriever may be directly measured by a stent retriever detection model that has trained a combination of the medical X-ray fluoroscopic image in which the stent retriever is imaged, and the lumen volume of the stent retriever in the image.

The size of the thrombus removal device has been described as the lumen volume of the stent retriever 313, but is not limited thereto. The size of the thrombus removal device may be, for example, a surface area of the stent retriever 313 present in the medical X-ray fluoroscopic image.

Referring back to FIG. 2, the second calculation function 354 calculates the change information indicating a change in size of the thrombus removal device from the plurality of medical X-ray fluoroscopic images. The second calculation function 354 is an example of a second calculation unit. More specifically, the second calculation function 354 calculates the change information indicating a change in the size of the thrombus removal device corresponding to a change in time series of the size of the thrombus removal device, from the sizes of the thrombus removal device appearing in the medical X-ray fluoroscopic images, calculated by the first calculation function 353.

The change in size of the thrombus removal device is change in lumen volume of the thrombus removal device. The change information includes a volume proportion of the thrombus removal device, and a time corresponding to the volume proportion of the thrombus removal device. The change information includes a difference in the volume proportion of the thrombus removal device between imaging, and a time corresponding to the difference in the volume proportion of the thrombus removal device between imaging.

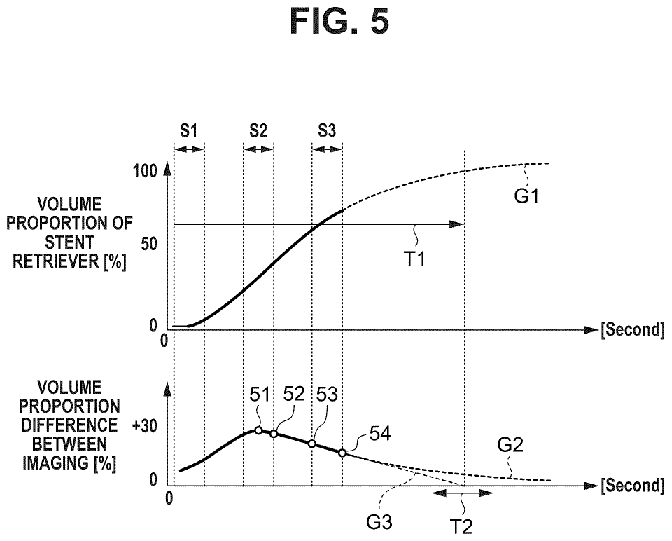

The change in size of the thrombus removal device is described with reference to FIG. 5. FIG. 5 is a graph illustrating an example of the change in size of the thrombus removal device according to the exemplary embodiment. FIG. 5 illustrates a graph G1 in which a lateral axis indicates a time [second], and a vertical axis indicates a volume proportion of the stent retriever [%]. In addition, FIG. 5 illustrates a graph G2 in which a lateral axis indicates a time [second], and a vertical axis indicates a volume proportion difference between imaging [%].

For example, after the stent retriever placed in the blood vessel of the subject is rapidly deployed to the inside of the blood vessel from a state where the lumen volume is close to 0%, the stent retriever expands while gradually engaging the thrombus. Outward deployment force of the stent retriever with elasticity of a material is referred to as axial force. When the axial force and force received from the thrombus or a blood vessel wall are balanced, the deployment of the stent retriever is completely stopped.

A volume proportion that is 100% in a case where the stent retriever is completely deployed in a space with no obstructions (in a state where no obstruction is present around the stent retriever), and is 0% in a case where the stent retriever is ideally and completely folded and the volume is zero is considered. When fluoroscopic imaging is intermittently repeated, in intermittent imaging sections from start of the imaging to a current time point, the change in the volume proportion of the stent retriever gradually engaging the thrombus, and the change in volume proportion difference between imaging frames are respectively obtained as the graph G1 and the graph G2.

Referring back to FIG. 2, the third calculation function 355 calculates the first time information indicating the time period during which the thrombus removal device is left in place in the subject, based on the change information. The third calculation function 355 is an example of a third calculation unit. More specifically, the third calculation function 355 calculates the first time information indicating the time period during which the thrombus removal device is placed in the subject, based on the change information calculated by the second calculation function 354.

The first time information indicates a time period starting from a timing when the thrombus removal device is placed in the subject, ending at a timing when the axial force and the force received from the thrombus or the blood vessel wall are balanced and the deployment of the thrombus removal device is regarded as being completely stopped. The first time information is an example of the recommended waiting time.

For example, the third calculation function 355 calculates, as the recommended waiting time, a time period from when the difference in the volume proportion of the stent retriever passes a peak to when the difference in the volume proportion becomes zero from a gradient of the difference in the volume proportion of the stent retriever.

The recommended waiting time is described with reference to FIG. 5. For example, in an imaging section S1 illustrated in FIG. 5, the difference in the volume proportion of the stent retriever increases. Therefore, the third calculation function 355 does not calculate the recommended waiting time. In an imaging section S2 illustrated in FIG. 5, the third calculation function 355 calculates a recommended waiting time T1 from the gradient of the difference between an observation time point 51 and an observation time point 52 on the latter half in which the difference in the volume proportion of the stent retriever reduces.

Further, in an imaging section S3 illustrated in FIG. 5, the difference in the volume proportion reduces over the entire stent retriever, so that the third calculation function 355 calculates the recommended waiting time T1 from the gradient of the difference (graph G3 illustrated in FIG. 5) using the observation time point 51, the observation time point 52, an observation time point 53, and an observation time point 54.

The above-described method is not restrictive. To improve the accuracy of the recommended waiting time T1, the third calculation function 355 may use observation data between the observation time point 51 and the observation time point 52, and/or observation data between the observation time point 53 and the observation time point 54. Alternatively, the third calculation function 355 may perform weighting (e.g., weighting in order of observation time point 51<observation time point 52<observation time point 53<observation time point 54) on the observation data newly observed to calculate the recommended waiting time T1.

The third calculation function 355 may calculate a value relating to reliability of the calculated recommended waiting time T1. For example, the third calculation function 355 may calculate a predicted section T2 with respect to time when the difference in the volume proportion becomes zero using linear regression. Instead of the linear regression based on only the gradient at a plurality of observation time points for changes in differences, nonlinear regression or curve-fitting may be applied. Alternatively, curve-fitting of a model expression previously created for the deployment of the stent retriever may be applied to the change in the volume proportion.

Referring back to FIG. 2, the output control function 356 performs control to output the first time information. The output control function 356 is an example of an output control unit. More specifically, the output control function 356 performs control to cause the display 340 to output the first time information calculated by the third calculation function 355.

For example, in a case where, after imaging in each of the imaging sections is finished, the third calculation function 355 calculates the recommended waiting time in any of the imaging sections, the output control function 356 outputs the calculated recommended waiting time to the display 340. Thereafter, in a case where the third calculation function 355 further calculates the recommended waiting time in another imaging section, the output control function 356 updates the recommended waiting time, and outputs the updated recommended waiting time to the display 340.

This process enables the operator to perform imaging again at a time point when the recommended waiting time has approached, check the state of the stent retriever, and retrieve the stent retriever entangled with the thrombus, without blindly waiting. Thus, the operator can avoid wasteful waiting. For example, in a case where a time period before the shape change in the stent retriever is completely stopped is defined as an ideal waiting time, and it is assumed that the shape change in the stent retriever is stopped by gradually weakening a degree of the shape change, the recommended waiting time becomes close to the ideal waiting time every time the imaging is intermittently performed and the recommended waiting time is updated. At a time point when the shape change in the stent retriever is completely stopped, the recommended waiting time can be regarded as being coincident with the ideal waiting time.

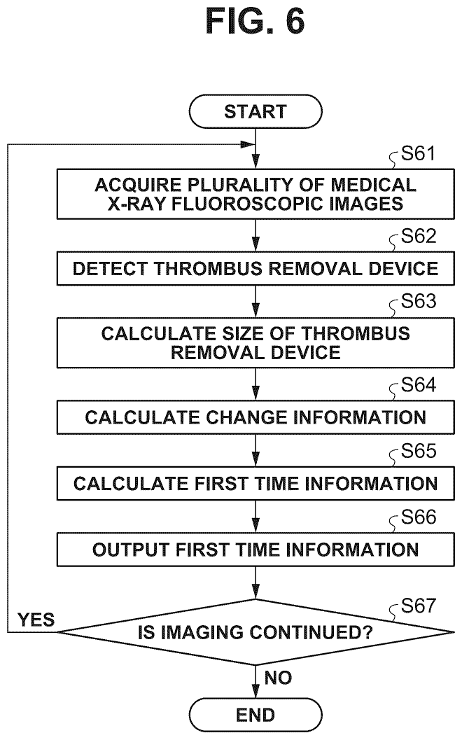

Next, processing to be performed by the medical image processing apparatus 300 is described. FIG. 6 is a flowchart illustrating processing procedure to be performed by the medical image processing apparatus 300 according to the exemplary embodiment. In FIG. 6, the processing is started at a time point when the operator inserts the thrombus removal device into the subject, and the thrombus device reaches a thrombus.

In step S61, the acquisition function 351 acquires the plurality of medical X-ray fluoroscopic images. In step S62, the detection function 352 detects, from each of the plurality of medical X-ray fluoroscopic images, the thrombus removal device that has been placed in the blood vessel of the subject and appears in the medical X-ray fluoroscopic image. In step S63, the first calculation function 353 calculates, from each of the plurality of medical X-ray fluoroscopic images, the size of the thrombus removal device that has been placed in the blood vessel of the subject and is present in the medical X-ray fluoroscopic image. In step S64, the second calculation function 354 calculates the change information indicating a change in size of the thrombus removal device from the plurality of medical X-ray fluoroscopic images.

In step S65, the third calculation function 355 calculates the first time information indicating the time period during which the thrombus removal device is left in place in the subject, based on the change information. In step S66, the output control function 356 performs control to output the first time information. In step S67, the acquisition function 351 determines whether the imaging is continued. Here, if the acquisition function 351 determines that the imaging is continued (YES in step S67), the processing proceeds to step S61. If the acquisition function 351 determines that the imaging is not continued (NO in step S67), the processing is ended.

As described above, the medical image processing apparatus 300 according to the exemplary embodiment calculates, from each of the plurality of medical X-ray fluoroscopic images, the size of the thrombus removal device that has been placed in the blood vessel of the subject and appears in the medical X-ray fluoroscopic image, calculates the change information indicating a change in the size from the plurality of medical X-ray fluoroscopic images, calculates the first time information indicating the time period during which the thrombus removal device is left in place in the subject, based on the change information, and outputs the first time information.

According to at least one of the exemplary embodiments described above, for example, in a case where the operator retrieves the thrombus present in the blood vessel of the subject by using the stent retriever, the medical image processing apparatus 300 provides the recommended waiting time to the operator, based on the change in the size of the thrombus removal device appearing in the plurality of medical X-ray fluoroscopic images.

The operator grasps the recommended waiting time. Thus, without blindly waiting, the operator can perform imaging again at a time point when the recommended waiting time has approached, and check the deployment state of the stent retriever. As a result, the operator can retrieve the stent retriever with a check of the deployment state of the stent retriever, thus reducing wasteful waiting.

The above-described exemplary embodiment can be appropriately modified and implemented by changing a part of the components or the functions included in each of the apparatuses. Therefore, in the following, some modifications according to the above-described exemplary embodiment are described as other exemplary embodiments. In the following, differences from the above-described exemplary embodiment are mainly described, and points common to the above-described configurations are denoted by the same reference numerals, and detailed description of the contents is omitted. The other exemplary embodiments described below may be individually implemented, or may be implemented in combination as appropriate.

### First Modification

In the above-described exemplary embodiment, a form in which the third calculation function 355 of the processing circuitry 350 calculates the first time information indicating the time period during which the thrombus removal device is left in place in the subject, based on the change information is described; however, this is not restrictive. For example, the third calculation function 355 of the processing circuitry 350 calculates a volume proportion at a time point when the recommended waiting time has elapsed, by adding the predicted volume proportion differences.

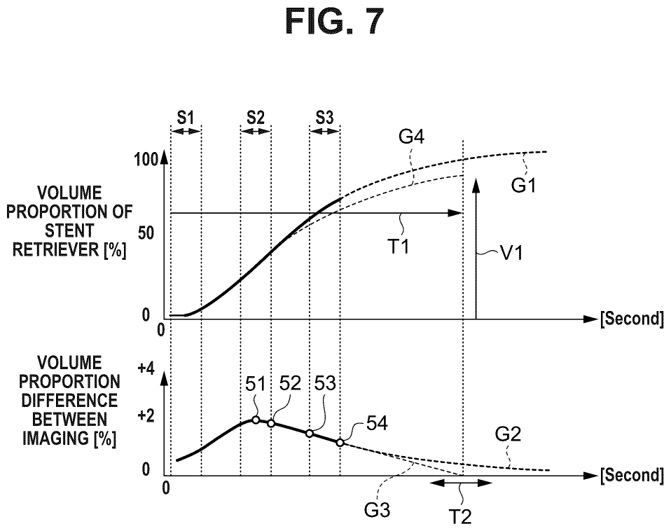

FIG. 7 is a graph illustrating an example of a change in size of a thrombus removal device according to a first modification. FIG. 7 illustrates, in addition to the graphs illustrated in FIG. 5, a graph G4 indicating the volume proportion of the stent retriever [%] with the observation time point 51 as a base point, and an integral value V1 obtained by adding the predicted volume proportion differences. More specifically, the third calculation function 355 calculates the volume proportion at the time point when the recommended waiting time T1 has elapsed, by adding differences in the volume proportion of the stent retriever between imaging.

For example, as illustrated in FIG. 7, the third calculation function 355 calculates the volume proportion at the time point when the recommended waiting time T1 has elapsed, by adding differences in the volume proportion of the stent retriever at the observation time point 51, the observation time point 52, the observation time point 53, and the observation time point 54. Thus, the operator can grasp the deployment state of the stent retriever.

### Second Modification

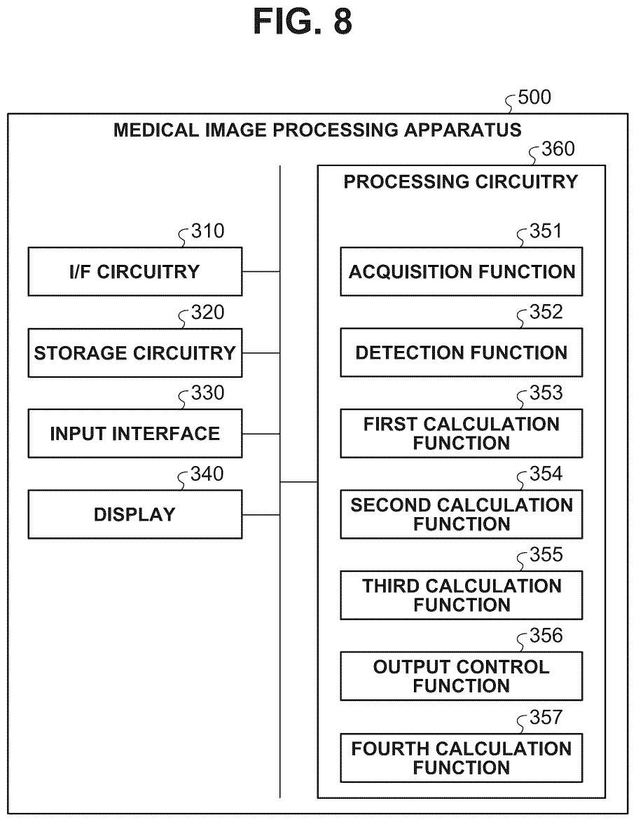

For example, the processing circuitry of the medical image processing apparatus may calculate and output a next imaging timing suitable for the recommended waiting time calculated by the third calculation function 355. FIG. 8 is a diagram illustrating an exemplary configuration of a medical image processing apparatus 500 according to a second modification. Processing circuitry 360 of the medical image processing apparatus 500 according to the second modification performs the acquisition function 351, the detection function 352, the first calculation function 353, the second calculation function 354, the third calculation function 355, the output control function 356, and a fourth calculation function 357.

The fourth calculation function 357 calculates a next imaging timing suitable for the recommended waiting time. The fourth calculation function 357 is an example of a fourth calculation unit. More specifically, the fourth calculation function 357 calculates the next imaging timing suitable for the recommended waiting time calculated by the third calculation function 355.

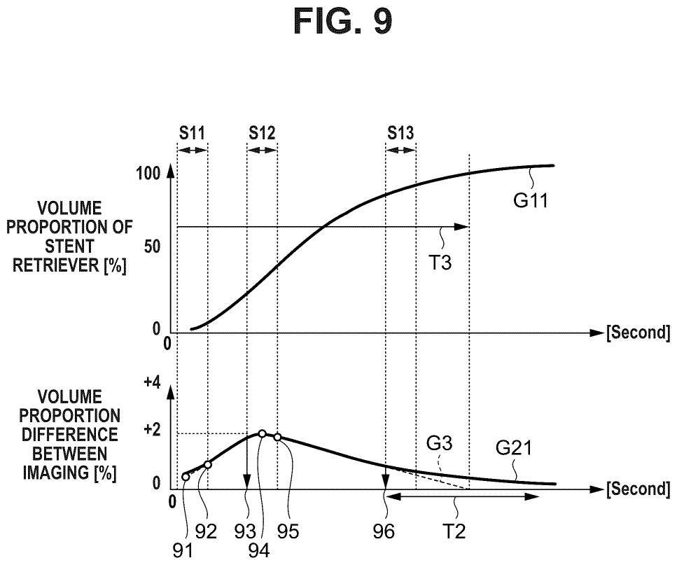

The imaging timing is described with reference to FIG. 9. FIG. 9 is a graph illustrating an example to explain the imaging timing according to the second modification. FIG. 9 illustrates a graph G11 in which a lateral axis indicates a time [second] and a vertical axis indicates a volume proportion of the stent retriever [%]. In addition, FIG. 9 illustrates a graph G21 in which a lateral axis indicates a time [second] and a vertical axis indicates a volume proportion difference between imaging [%]. FIG. 9 also illustrates a recommended waiting time T3 calculated by the third calculation function 355.

The fourth calculation function 357 calculates a time when a difference in the volume proportion reaches a value that is drastic for a change for the stent retriever (an example of such a value is +2%), from results at an observation time point 91 and an observation time point 92 in the graph G21 in an imaging section S11, and calculates an imaging timing 93 in an imaging section S12 at which imaging is recommended, from the calculated time. The output control function 356 performs control to cause the display 340 to output the imaging timing 93 calculated by the fourth calculation function 357.

The fourth calculation function 357 calculates a time when a difference in the volume proportion reaches a predicted section T2, from results at an observation time point 94 and an observation time point 95 in the graph G21 in the imaging section S12, and calculates an imaging timing 96 in an imaging section S13 at which imaging is recommended, from the calculated time. The output control function 356 controls the display 340 to output the imaging timing 96 calculated by the fourth calculation function 357. As a result, the operator can grasp the imaging timing, thus reducing an exposure dose to the subject.

### Third Modification

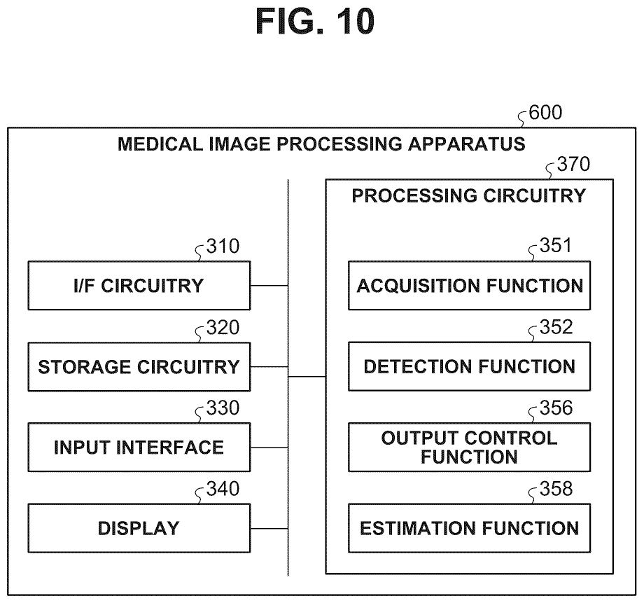

For example, the processing circuitry of the medical image processing apparatus may detect, from each of the plurality of acquired medical X-ray fluoroscopic images, the thrombus removal device that has been placed in the blood vessel of the subject and appears in the medical X-ray fluoroscopic image, and calculate and output second time information indicating a time period during which the thrombus removal device is placed in the subject. FIG. 10 is a diagram illustrating an exemplary configuration of a medical image processing apparatus 600 according to a third modification. Processing circuitry 370 of the medical image processing apparatus 600 according to the third modification performs the acquisition function 351, the detection function 352, the output control function 356, and an estimation function 358.

The estimation function 358 estimates, from the plurality of medical X-ray fluoroscopic images, second time information indicating a time period during which the thrombus removal device that has been placed in the blood vessel of the subject and is present in the medical X-ray fluoroscopic images is placed. The estimation function 358 is an example of an estimation unit. More specifically, when the detection function 352 detects, from each of the plurality of medical X-ray fluoroscopic images acquired by the acquisition function 351, the thrombus removal device that has been placed in the blood vessel of the subject and appears in the medical X-ray fluoroscopic image, the estimation function 358 estimates the size of the thrombus removal device.

The estimation function 358 estimates the change information indicating a change in the size of the thrombus removal device corresponding to a change in time series of the size of the thrombus removal device. The estimation function 358 further estimates the second time information indicating the time period during which the thrombus removal device is left in place in the subject, based on the estimated change information.

The output control function 356 performs control to output the second time information. The second time information is information indicating a time period starting from a timing when the thrombus removal device is placed in the subject ending at a timing when the axial force and the force received from the thrombus or the blood vessel wall are balanced and the deployment of the thrombus removal device is regarded as being completely stopped. The second time information is an example of the recommended waiting time.

According to at least one of the modifications described above, for example, in a case where the operator retrieves a thrombus present in the blood vessel of the subject by using the thrombus removal device, the medical image processing apparatus 600 estimates, from the plurality of medical X-ray fluoroscopic images, the recommended waiting time indicating the time period during which the thrombus removal device that has been placed in the blood vessel of the subject and appears in the medical X-ray fluoroscopic image is placed, and outputs the recommended waiting time. The operator can grasp the recommended waiting time. Thus, without blindly waiting, the operator can perform imaging again at a time point when the recommended waiting time has approached, and check the deployment state of the stent retriever. Thus, the operator can retrieve the stent retriever, which makes it possible to reduce wasteful waiting.

### Application to Artery of Lower Limb

In the above-described exemplary embodiments, the blood vessel described in the above exemplary embodiment is described as a cerebral blood vessel; however, the blood vessel is not limited thereto, and the exemplary embodiments are applicable to a thrombus in an artery of a lower limb. In other words, the operator can grasp a timing when the thrombus in the artery of the lower limb is to be retrieved, by using the recommended waiting time calculated by the processing circuitry of the medical image processing apparatus.

In the above-described exemplary embodiments, the example in the case where the processing functions are realized by a single piece of processing circuitry (processing circuitry 350, processing circuitry 360, and processing circuitry 370) is described; however, the exemplary embodiments are not limited thereto. For example, each of the processing circuitry 350, the processing circuitry 360, and the processing circuitry 370 may be configured by a combination of a plurality of independent processors, and the processors may execute corresponding programs to realize the respective processing functions. The processing functions included in each of the processing circuitry 350, the processing circuitry 360, and the processing circuitry 370 may be realized by being appropriately distributed or integrated into a single or plurality pieces of processing circuitry.

In a case where the technical idea according to the exemplary embodiments is implemented by a medical image processing method, the medical image processing method includes calculating, from each of the plurality of medical X-ray fluoroscopic images, the size of the thrombus removal device that has been placed in the blood vessel of the subject and appears in the medical X-ray fluoroscopic image, calculating the change information indicating a change in size from the plurality of medical X-ray fluoroscopic images, calculating the first time information indicating the time when the thrombus removal device is to be retrieved from the subject, based on the change information, and performing control to output the first time information. A processing procedure with the medical image processing method and advantageous effects thereof are similar to those in the exemplary embodiments. Thus, a description thereof is omitted.

In a case where the technical idea according to the exemplary embodiments is implemented by a medical image processing program, the medical image processing program causes a computer to realize calculating, from each of the plurality of medical X-ray fluoroscopic images, the size of the thrombus removal device that has been placed in the blood vessel of the subject and appears in the medical X-ray fluoroscopic image, calculating the change information indicating a change in size from the plurality of medical X-ray fluoroscopic images, calculating, based on the change information, the first time information indicating the time when the thrombus removal device is to be retrieved from the subject and performing control to output the first time information. A processing procedure by the medical image processing program and effects thereof are similar to those in the exemplary embodiments. Thus, a description thereof is omitted.

The term “processor” used in the description of the foregoing exemplary embodiments refers to a circuit such as a central processing unit (CPU), a graphics processing unit (GPU), an application specific integrated circuit (ASIC), and a programmable logic device (e.g., a simple programmable logic device [SPLD], a complex programmable logic device [CPLD], or a field programmable gate array [FPGA]). Instead of storing the programs in the storage circuit, the programs may be directly built in the processor circuit. In such a case, the processor implements the functions by reading the built-in programs in its own circuit and executing the programs. The processors according to the exemplary embodiments are not limited to a single-circuit configuration. A plurality of independent circuits may be combined into a processor that implements the functions.

The program to be executed by the processor is provided by being previously incorporated in a read only memory (ROM), a storage unit, or the like. The program may be provided by being stored in a computer-readable storage medium such as a compact disk (CD)-ROM, a flexible disk (FD), a CD-recordable (CD-R), and a digital versatile disk (DVD), as a file in a format that can be installed in or executed by the apparatus.

The program may be stored in a computer connected to a network such as the Internet, and may be provided or distributed by being downloaded through the network. For example, the program includes modules including functional units described below. In terms of actual hardware, a CPU reads the processing program from a storage medium such as a ROM and executes the program, whereby the modules are loaded into and generated on a main storage device.

According to the at least one exemplary embodiment described above, it is possible to provide a recommended waiting time of a thrombus removal device in a blood vessel of a subject in accordance with a case.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising processing circuitry configured to:
calculate, from a plurality of medical X-ray fluoroscopic images, a size of a thrombus removal device that has been placed in a blood vessel of a subject and appears in the respective medical X-ray fluoroscopic images;
calculate change information indicating a change in the size from the plurality of medical X-ray fluoroscopic images;
calculate first time information indicating a time period during which the thrombus removal device is left in place in the subject, based on the change information; and
perform control to output the first time information.

2. The medical image processing apparatus according to claim 1, wherein the change in the size is a change in a lumen volume of the thrombus removal device.

3. The medical image processing apparatus according to claim 1, wherein the first time information is information indicating a time period from a timing when the thrombus removal device is placed in the subject as a starting point to a timing when deployment of the thrombus removal device is regarded to be completely stopped as an ending point.

4. A medical image processing method comprising:
calculating, from a plurality of medical X-ray fluoroscopic images, a size of a thrombus removal device that has been placed in a blood vessel of a subject and appears in the respective medical X-ray fluoroscopic images;
calculating change information indicating a change in the size from the plurality of medical X-ray fluoroscopic images;
calculating first time information indicating a time when the thrombus removal device is to be retrieved from the subject, based on the change information; and
performing control to output the first time information.

5. A non-transitory computer-readable storage medium storing a program for causing a computer to:
calculate, from a plurality of medical X-ray fluoroscopic images, a size of a thrombus removal device that has been placed in a blood vessel of a subject and appears in the respective medical X-ray fluoroscopic images;
calculate change information indicating a change in the size from the plurality of medical X-ray fluoroscopic images;
calculate first time information indicating a time when the thrombus removal device is to be retrieved from the subject, based on the change information; and
perform control to output the first time information.
